(19) United States
(12) Patent Application Publication
(10) Pub. No.: US 2026/0196329 A1
(43) Pub. Date: Jul. 9, 2026

(54) System for providing customized information and coaching for cancer patients

(71) Applicant: CHUNG-ANG UNIVERSITY INDUSTRY-ACADEMIC COOPERATION FOUNDATION, Seoul (KR)

(72) Inventors: Sun Mi KIM, Seoul (KR); Doug Hyun HAN, Seoul (KR); Eun Seung YU, Gyeonggi-do (KR); Hee Jun KIM, Seoul (KR)

(73) Assignee: CHUNG-ANG UNIVERSITY INDUSTRY-ACADEMIC COOPERATION FOUNDATION, Seoul (KR)

(21) Appl. No.: 19/453,745

(22) Filed: Jan. 20, 2026

(30) Foreign Application Priority Data
Nov. 21, 2024 (KR) 10-2024-0167345

(51) Int. Cl. G16H 20/30; A63B 24/00; G09B 5/02; G16H 20/60; G16H 20/70

(52) U.S. Cl. CPC G16H 20/30; A63B 24/0075; G09B 5/02; G16H 20/60; G16H 20/70

(57) ABSTRACT

The present invention relates to a system for providing customized information and coaching for cancer patients. According to the present invention, in the system for providing customized information and coaching for cancer patients, it can comprise a user device that inputs and manages user information from users, provides content and self-management methods to users based on the user information, and designs users' wellness life; and a management server that manages multiple users' information, monitors each user's psychological state, health status, exercise, and rehabilitation training, and manages the content provided to users, wherein the user device and management server may be a wireless-connected. Accordingly, the present invention can improve the treatment outcomes of cancer patients by providing alerts for hospital visits, medication intake, and other activities.

### TECHNICAL FIELD

The present invention relates to a system for providing customized information and coaching for cancer patients, more specifically, it relates to the system for providing customized information and coaching for cancer patients that provides at least one of the following functions to survivors of common cancers (Colorectal cancer, Lung cancer, Breast cancer, Thyroid cancer): personalized schedule management, provision of cancer-related information, coaching to promote healthy behaviors, and wellness life design.

This application claims priority based on Korean Patent Application No. 10-2024-0167345 filed on Nov. 21, 2024, and the entire contents of the specification and figures disclosed in the application are incorporated herein by reference.

### BACKGROUND OF THE INVENTION

Common cancers are cancers with a high occurrence rate. In 2021, the common cancers occurring in South Korea were thyroid cancer (12.7%), colorectal cancer (11.8%), lung cancer (11.4%), stomach cancer (10.6%), and breast cancer (10.4%), accounting for over 50% of all cases.

For patients requiring long-term medication, such as cancer patients, the dates for inpatient treatment are not regular, as they depend on the hospital's number of scheduled inpatients and the number of beds allocated to each outpatient department and schedule fluctuations occur, such as additional pre-treatment examinations scheduled at regular intervals or treatment schedule changes due to medication supply instability, or problems like missing regularly scheduled medications also arise, necessitating schedule management for cancer patients.

Furthermore, for cancer patients, unverified information about cancer patients is publicly available online. To prevent further confusion among patients, it is necessary to provide information on treatment methods by cancer type and stage, along with details on managing associated side effects.

Additionally, according to data provided by Asan Medical Center in Seoul on Apr. 15, 2022, cancer patients are 4 to 10 times more likely to develop depression than the general population. The stages of psychological response in cancer patients, as outlined by the National Cancer Information Center, are divided into Stage 1 (Shock, Anxiety, Denial phase), Stage 2 (Reactive Depression phase), Stage 3 (Optimistic phase), and Stage 4 (Religious-Philosophical Turn phase) and psychological support programs are needed to help patients transition to the optimistic phase and the religious-philosophical phase.

Additionally, a program is needed to manage and correct the lifestyle habits of cancer patients.

### DETAILED DESCRIPTION OF THE INVENTION

### Technical Problem

Accordingly, the present invention is to provide a system for providing customized information and coaching for cancer patients that provides at least one of the following functions to survivors of common cancers (Colorectal cancer, Lung cancer, Breast cancer, Thyroid cancer): personalized schedule management, provision of cancer-related information, coaching to promote healthy behaviors, and wellness life design.

### Technical Solution

According to an embodiment of the present invention for achieving these technical challenges, in system for providing customized information and coaching for cancer patients, it can comprise a user device that inputs and manages user information from users, provides content and self-management methods to users based on the user information, and designs users' wellness life; and a management server that manages multiple users' information, monitors each user's psychological state, health status, exercise, and rehabilitation training, and manages the content provided to users, wherein the user device and management server may be a wireless-connected.

Wherein the user device, can comprises a customer management module that inputs and stores user information and the user's cancer treatment schedule from the user; a content provision module that applies the user information and the user's cancer treatment schedule to a pre-established guide algorithm, considers the common cancer types and treatment timing, and provides content related to at least one of disease, treatment, or side effects; a coaching module that provides pre-set types of health information based on the user information, assesses the user's health status through pre-set multiple performance evaluation metrics, and provides self-management methods; and a wellness life design module that provides information on linked institutions within a pre-set radius based on the user's location information and provides dietary information based on the user information.

Wherein the user information, can comprise the user's personal information, the user's common cancer information, and the user's medical information, wherein the cancer treatment schedule comprises appointment schedules, test schedules, and medication routines, wherein the user's personal information comprises the user's name, age, gender, and mobile phone number, wherein the user's common cancer information comprises the type of common cancer type, stage of progression, the user's considerations, content viewing period, treatment timing, and the user's areas of interest, wherein the user's medical information comprises the user's hospital visit history, prescription history, and surgical history.

Wherein the customer management module, can comprise a customer information storage unit that inputs and stores the user information and the user's cancer treatment schedule from the user; and a schedule management unit that provides alerts to the user based on the user information and the user's cancer treatment schedule.

Wherein the content provision module, can provide users with the common cancer guidelines initiated on the server and content composed of data directly created by evidence-based practices from psychological experts and dietary specialists in the fields of internal medicine, surgery, psychiatry, and psycho-oncology.

Wherein the coaching module, can evaluate the user's psychological state as one of combat-type, anxious-type, compliance-type, suppression-type or self-abandonment-type using a psychological state assessment scale based on the Mental Adjustment to Cancer Scale (Mini-MAC), and can provide pre-set psychological coaching according to the assessed psychological state.

Wherein the coaching module, can provide appropriate exercise and rehabilitation training based on the user's health status and common cancer types.

Wherein the coaching module, can provide exercise and rehabilitation training focused on: pelvic floor muscle exercises for colorectal cancer, breathing exercises and chest exercises for lung cancer, shoulder and neck exercises for thyroid cancer, and upper limb exercises for breast cancer.

Wherein the coaching module, can assess the user's health status as one of the beginner, intermediate, or advanced using pre-set multiple performance evaluation metric, can provide Range of Motion (ROM) exercises or Active Assistive Range of Motion (AAROM) exercises for users with beginner health status, can provide Isometric exercises for users with intermediate health status, can provide Strengthening exercises and Stretching exercises for users with advanced health status.

Wherein the management server, can comprise a monitoring module that manages comprehensive user information by receiving at least one of the following inputs: user information, user's psychological state, user's health status, user's sleep status, user's activity data, user's dietary habits, and user's exercise and rehabilitation training history; and a content management module that uploads content to be provided to users and modifies or deletes uploaded content.

### Advantageous Effect

Accordingly, the present invention can improve treatment outcomes for cancer patients by providing alerts for hospital visits, medication intake, and other activities.

Furthermore, by periodically assessing the psychological state of cancer patients and providing corresponding coping strategies, adaptive coping with cancer can be fostered.

Additionally, by applying the patient's responses regarding sleep, dietary habits, physical activity, psychological state, and exercise to a pre-trained algorithm, personalized treatment guidance for cancer patients can be provided.

### BRIEF DESCRIPTION OF THE DRAWINGS

FIG. 1 is a schematic diagram of a system for providing customized information and coaching for cancer patients according to an embodiment of the present invention.

FIG. 2 is a schematic diagram of a user device according to an embodiment of the present invention.

FIG. 3 is a diagram illustrating an example of inputting the user's common cancer information according to an embodiment of the present invention.

FIG. 4 is a diagram illustrating an example of inputting the cancer treatment schedule according to an embodiment of the present invention.

FIG. 5 is a diagram illustrating an example of managing the user's cancer treatment schedule according to an embodiment of the present invention.

FIG. 6 is a diagram illustrating a content provision module according to an embodiment of the present invention.

FIGS. 7a and 7b are diagrams illustrating an example of evaluating a user's psychological state according to an embodiment of the present invention.

FIG. 8 is a diagram illustrating an example of determining a user's health status according to an embodiment of the present invention.

FIGS. 9a and b are diagrams illustrating an example of providing exercise and rehabilitation training according to an embodiment of the present invention.

FIG. 10 is a diagram illustrating an example of providing information on linked institutions according to an embodiment of the present invention.

FIG. 11 is a schematic diagram of a management server according to an embodiment of the present invention.

FIG. 12 is a diagram illustrating an example of monitoring a user's health coaching history according to an embodiment of the present invention.

FIG. 13 is a diagram illustrating an example of managing cancer information guide history according to an embodiment of the present invention.

### BEST MODE FOR CARRYING OUT THE INVENTION

The preferred embodiments of the present invention will now be described in detail with reference to the accompanying figures. In this process, the thickness of lines or the size of components shown in the figures can be exaggerated for clarity and convenience.

Throughout the specification, when any part is described as “comprising” a component, this means that, unless specifically stated otherwise, it does not exclude other components but can include additional components.

Furthermore, the terms described below are defined considering their function within the present invention and can vary depending on the user's and operator's intent or convention. Therefore, the definitions of these terms should be based on the context throughout this specification.

In the embodiments described below, the system for providing customized information and coaching for cancer patients is performed by a computing device comprising one or more processors and one or more memories capable of performing the following processes. Common cancers include breast cancer, colorectal cancer, lung cancer, and thyroid cancer, specific examples are provided for illustration.

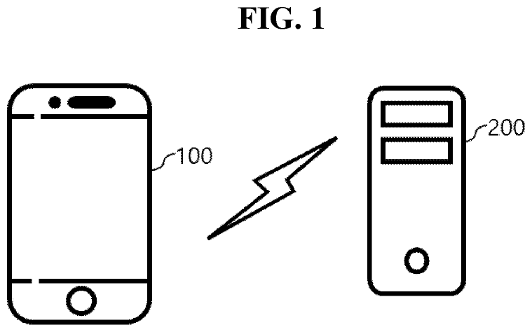

FIG. 1 is a schematic diagram of a system for providing customized information and coaching for cancer patients according to an embodiment of the present invention.

As shown in FIG. 1, the system for providing customized information and coaching for cancer patients may comprise a user device (100) (e.g., smartphone, tablet PC, laptop, etc.) and a management server (200), with the user device (100) and management server (200) connected wirelessly. At this time, the wireless communication method between the user device (100) and the management server (200) can comprise various data communication networks such as Beacon, Wi-Fi, Bluetooth, Zigbee, Infrared data association (IrDA), Ultra-wideband (UWB), Radio frequency identification (RFID), Local area network (LAN), Metropolitan area network (MAN), Wide area network (WAN), internet, 3G (third generation)/4G (fourth generation) mobile communication networks, Wireless Broadband Internet (WIBRO) or Long Term Evolution (LTE), etc.

First, the user device (100) that inputs and manages user information (For example, name, gender, age, mobile phone number, resident registration number, hospital visit information, etc.) entered by the user, provide content and self-management methods to users based on the user information, and can design users' wellness life.

Additionally, the user device (100) can download the customized information and coaching application for cancer patients via pre-set paths (e.g., QR codes, Play Store, App Store, etc.).

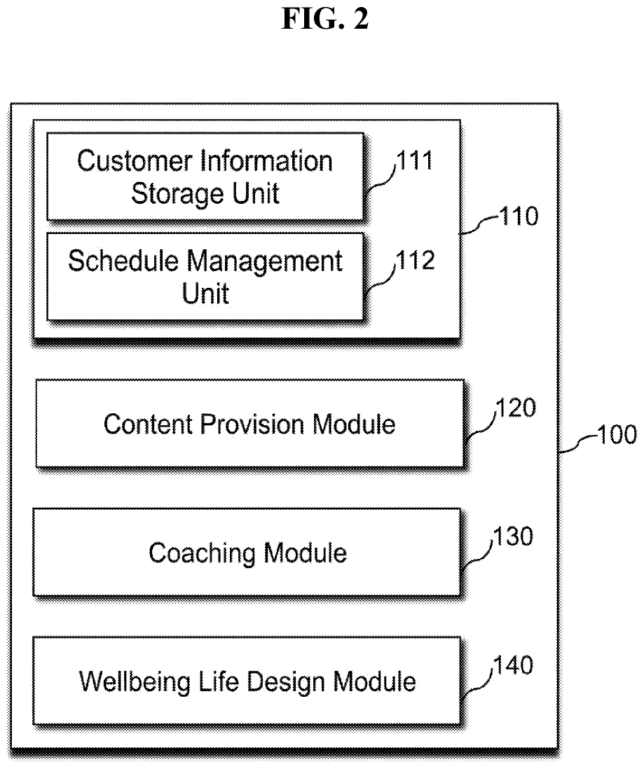

FIG. 2 is a schematic diagram of a user device according to an embodiment of the present invention.

As shown in FIG. 2, the user device (100) can comprise a customer management module (110), a content provision module (120), a coaching module (130), and a wellness life design module (140).

Specifically, the customer management module (110) can input and manage user information and the user's cancer treatment schedule (e.g., medical appointments, test schedules, medication routines, etc.) from the user and provide alerts.

Additionally, the customer management module (110) can manage the user's schedule in conjunction with the coaching module (130) and the Wellness life design module (140).

A customer information storage unit (111) can input and store user information and the user's cancer treatment schedule from the user. At this time, the user information comprises the user's personal information (name, age, gender, mobile phone number, etc.), the user's common cancer information (e.g., common cancer type (one of breast cancer, colorectal cancer, lung cancer, or thyroid cancer), stage of progression, the user's considerations, content viewing period, the user's areas of interest, etc.), and the user's medical information (e.g., the user's hospital visit history, prescription history, surgery history, etc.).

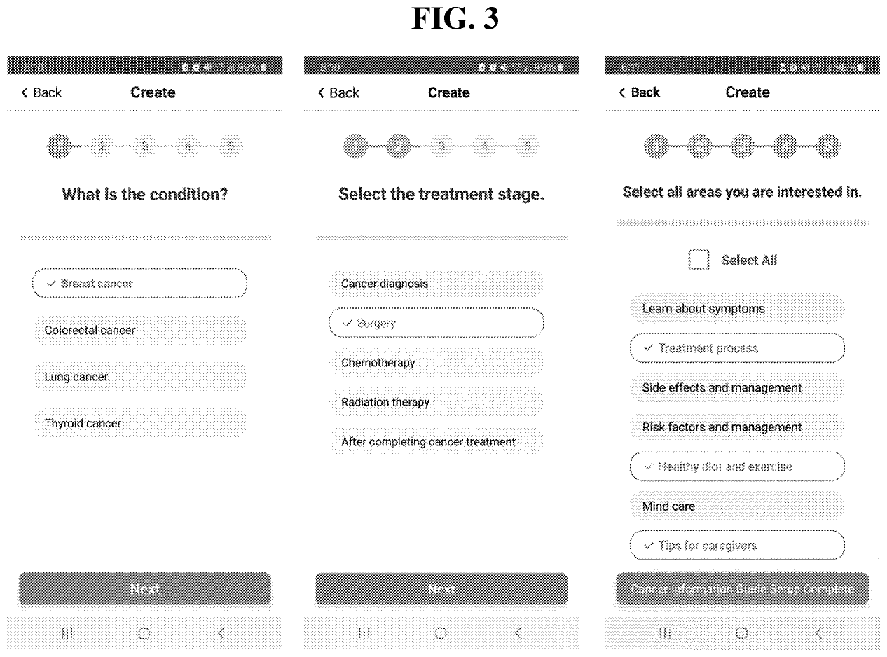

FIG. 3 is a diagram illustrating an example of inputting the user's common cancer information according to an embodiment of the present invention.

As shown in FIG. 3, the customer information storage unit (111) can input a setting signal from the user regarding any one of the following: the type of the user's common cancer, the stage of progression of the common cancer, the timing of treatment for the common cancer, the user's considerations (e.g., taking diuretic medication due to hypertension, using an inhaler due to respiratory disease), the content viewing period (e.g., either 60 days or 90 days), and the user's area of interest, and store it as customer information.

Additionally, the customer information storage unit (111) can input and store the user's cancer treatment schedule (e.g., hospital appointment dates, medication schedules, examination schedules) as customer information.

According to one embodiment of the present invention, the customer information storage unit (111) can input prescription details for the user from the server of a medical institution (e.g., the hospital visited by the patient, the National Health Insurance Service, etc.) and store them as customer information.

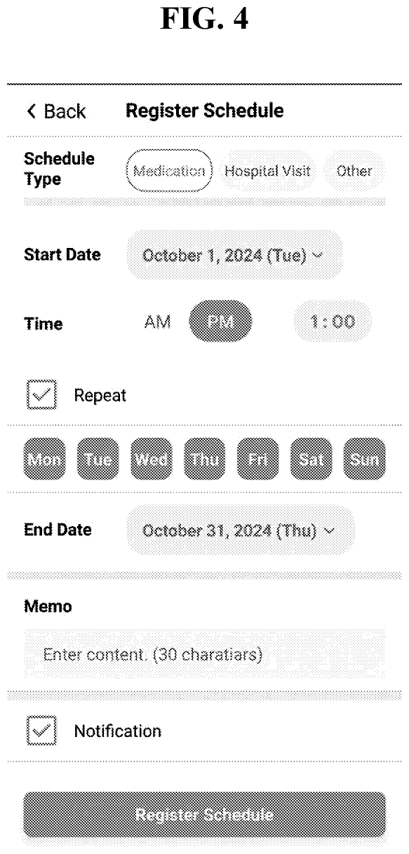

FIG. 4 is a diagram illustrating an example of inputting the cancer treatment schedule according to an embodiment of the present invention.

As shown in FIG. 4, the customer information storage unit (111) can input from the user regarding at least one of the following: the user's hospital appointment date, medication schedule (e.g., inhaling Foster twice daily at 07:00, taking Ventolin in emergencies, taking Singulair tablets daily at 19:00, etc.), or reminder notes (e.g., blood pressure medication, etc.), and store them as customer information.

A schedule management unit (112) can provide alerts to the user based on the stored user information and the user's cancer treatment schedule.

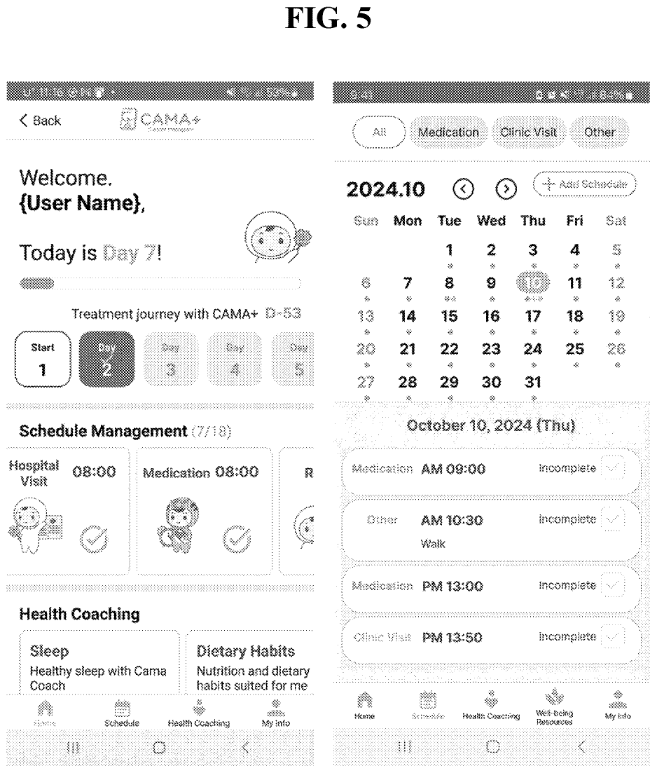

FIG. 5 is a diagram illustrating an example of managing the user's cancer treatment schedule according to an embodiment of the present invention.

As shown in FIG. 5, the Schedule Management Unit (112) can provide the user's treatment date (e.g., “Today is Day 27th!”) or provide the user's stored cancer treatment schedule (e.g., hospital appointment dates, medication schedules, test schedules), and/or provide alerts based on the user's cancer treatment schedule.

According to one embodiment of the present invention, the schedule management unit (112) can assist the user in taking medication by providing an alarm to the user according to the stored medication schedule.

Additionally, the schedule management unit (112) can manage the user's designed wellness life schedule, psychological training schedule, and coaching schedule.

Through this, the present invention can motivate the user to manage schedules for key self-care activities and support enhanced continuous management and treatment adherence through habit formation.

The content provision module (120) can apply user information and the user's cancer treatment schedule (e.g., medical appointments, test schedules, medication routines, etc.) to a pre-established guide algorithm to provide content related to at least one of disease, treatment, or side effects, categorized by common cancer types (e.g., breast cancer, thyroid cancer, colorectal cancer, lung cancer, common) and treatment timing (e.g., at diagnosis, chemotherapy, radiation therapy, surgery, etc.).

At this time, the content is composed of guidelines for common cancers published on the server (e.g., medical institution or National Cancer Center websites, medical journal databases, etc.), data directly authored by evidence-based experts in internal medicine, surgery, psychiatry, psychosocial oncology, and nutrition, and comprises pre-set types (side effects, coping strategies, risk factors, management methods, symptoms, treatment process, assessing patient psychological state, tips for caregivers, healthy diet and exercise, etc.) of information such as text, card news, and videos, etc.

Here, the guide algorithm can be structured to organize content by date according to the timing (e.g., immediately after diagnosis, before surgery, after surgery, before chemotherapy, after chemotherapy, after all treatments are completed, etc.) and priority (e.g., healthy diet and exercise (1st priority), treatment process (2nd priority), management methods (3rd priority), coping strategies (4th priority), side effects (5th priority), risk factors (6th priority), symptoms (7th priority), tips for caregivers (8th priority), etc.) of treatment for each common cancer type (e.g., breast cancer, colorectal cancer, lung cancer, thyroid cancer)

According to one embodiment of the present invention, when the common cancer is lung cancer and the treatment timing is preoperative, the guide algorithm can sequentially organize content classified by the server as information for lung cancer patients undergoing preoperative treatment into priority levels such as 1st priority and 2nd priority (Day 1), 3rd priority (Day 2), 4th priority (Day 3), 5th priority (Day 4), 6th priority (Day 5), 7th and 8th priorities (Day 6), 1st to 3rd priorities (Day 7).

Additionally, the content provision module (120) can convert text within the content to speech, adjust the text size within the content to a pre-set level (e.g., 7 levels), or designate it as favorite content, depending on the user's selection. In this case, the content provision module (120) can convert text within the content to speech using conventionally disclosed text-to-speech conversion technology (e.g., Text to Speech).

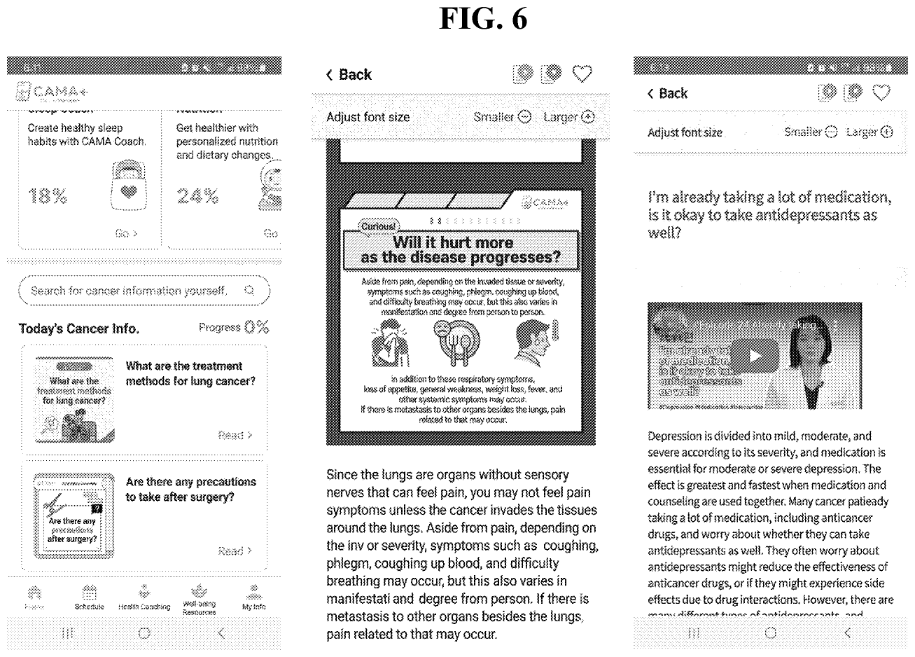

FIG. 6 is a diagram illustrating a content provision module according to an embodiment of the present invention.

As shown in FIG. 6, the content provision module (120) collects the user's usage time for each content type. Based on the user information, the user's cancer treatment schedule, and the usage time for each content type, it analyzes the correlation between cancer characteristics, user information, and content usage results. Through this, it can further train the guide algorithm to provide customized information to the user.

The coaching module (130) can provide pre-set types of health information (e.g., sleep, dietary habits, physical activity, psychological state, exercise, and rehabilitation training) based on the input user information. It can assess the user's health status using pre-set multiple functional performance evaluation scales and provide self-management methods.

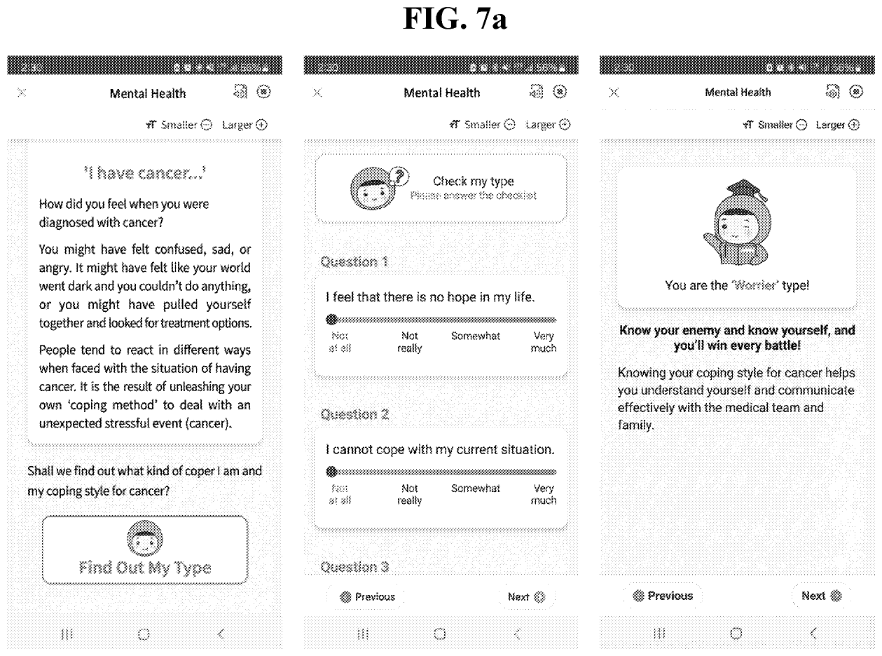

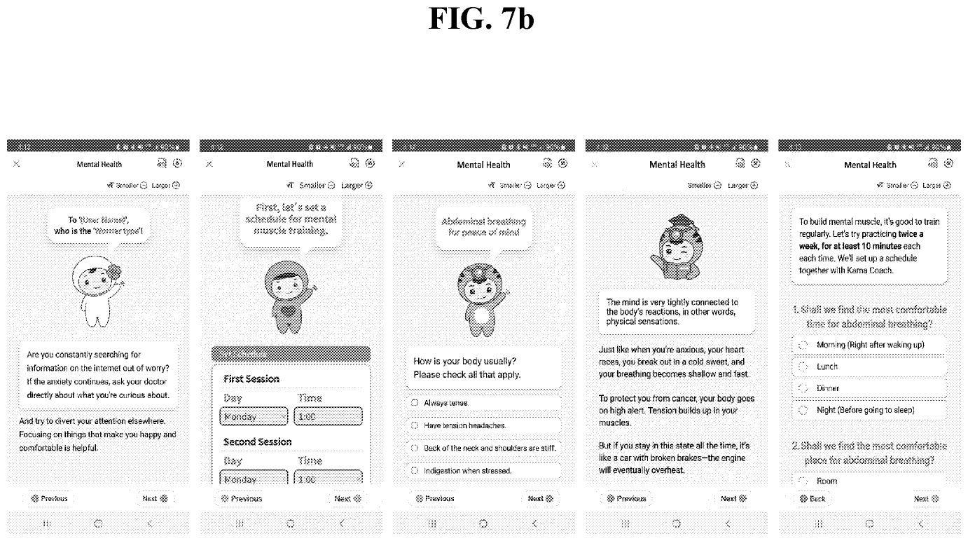

FIGS. 7a and 7b are diagrams illustrating an example of evaluating a user's psychological state according to an embodiment of the present invention.

As shown in FIG. 7a, the coaching module (130) can assess the user's psychological state using a psychological state evaluation scale based on the Mental Adjustment to Cancer Scale (Mini-MAC). In this process, each response (Not at all, Not really, Yes, Very much) is assigned a score ranging from 1 to 4 points and the user's psychological state can be assessed as follows: if the total score is below the first threshold score, it is the combat-type; if it is at or above the first threshold score but below the second threshold score, it is the anxious-type; if it is at or above the second threshold score but below the third threshold score, it is the compliance-type; if it is at or above the third threshold score but below the fourth threshold score, it is the, suppression-type; and if it is at or above the fourth threshold score, it is the or self-abandonment-type.

As shown in FIG. 7b, the coaching module (130) can provide pre-set psychological coaching based on the assessed psychological state. This psychological coaching may comprise stress recognition (fatigue for thyroid cancer; stoma for colorectal cancer; body image and sexual function for breast cancer; postoperative breathing discomfort and decreased physical function for lung cancer; anxiety about cancer recurrence and discomfort from anticancer treatment for common to all), learning coping strategies, relaxation training, meditation, and breathing techniques.

According to one embodiment of the present invention, the coaching module (130) can provide psychological coaching to enhance motivation and self-efficacy when the user's psychological state is self-abandonment-type, provide psychological coaching to reduce anxiety and manage stress when the user's psychological state is anxious-type, and provide psychological coaching for realistic acceptance when the user's psychological state is suppression-type.

Additionally, the coaching module (130) can collect the user's sleep status and activity data by linking with the health management application on the user device (100), and can receive input on the user's dietary habits based on the user's selection.

Additionally, the coaching module (130) can receive input from the user regarding their goals and degree of achievement, apply a coaching model pre-established based on the user information and goals, and provide a step-by-step coaching program. Here, the coaching model is a model trained to derive a coaching program when it receives input of user information, including the user's status, and the goals, using coaching programs tailored to multiple common cancer types and user states (for example, Stage 1, Stage 2, Stage 3, and Stage 4) as training data. Furthermore, the coaching model can be trained to provide different feedback based on the degree of achievement.

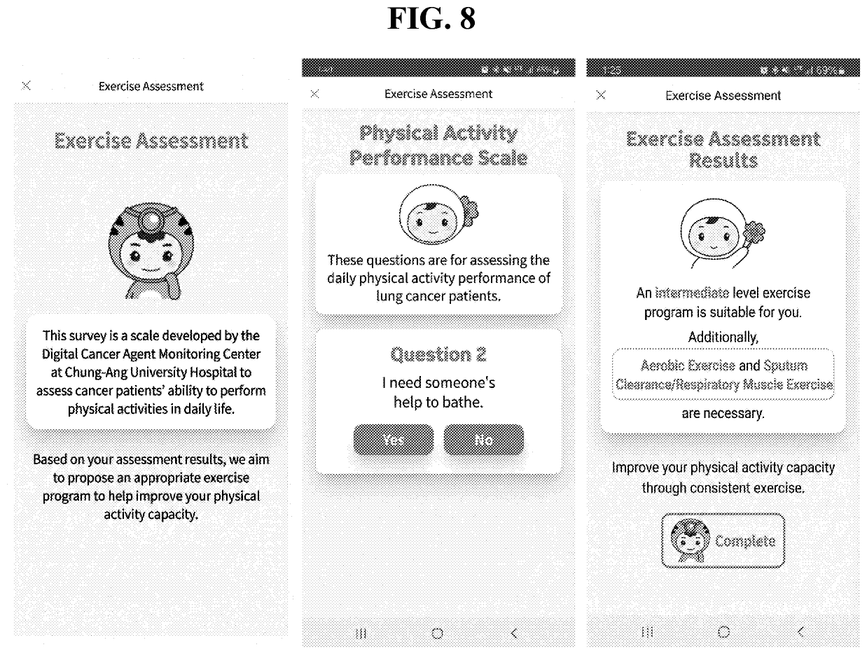

FIG. 8 is a diagram illustrating an example of determining a user's health status according to an embodiment of the present invention.

As shown in FIG. 8, the coaching module (130) can assess the user's health status as one of beginner, intermediate, or advanced using pre-set multiple performance evaluation metric. At this time, the performance evaluation metric consists of 13 common questions and 3 or 4 questions specific to each common cancer type.

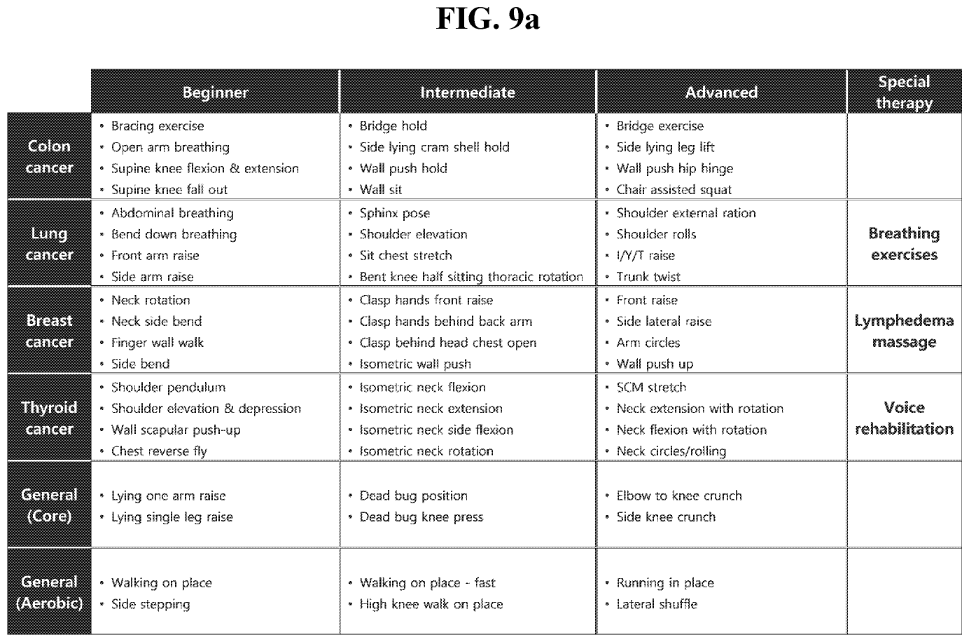

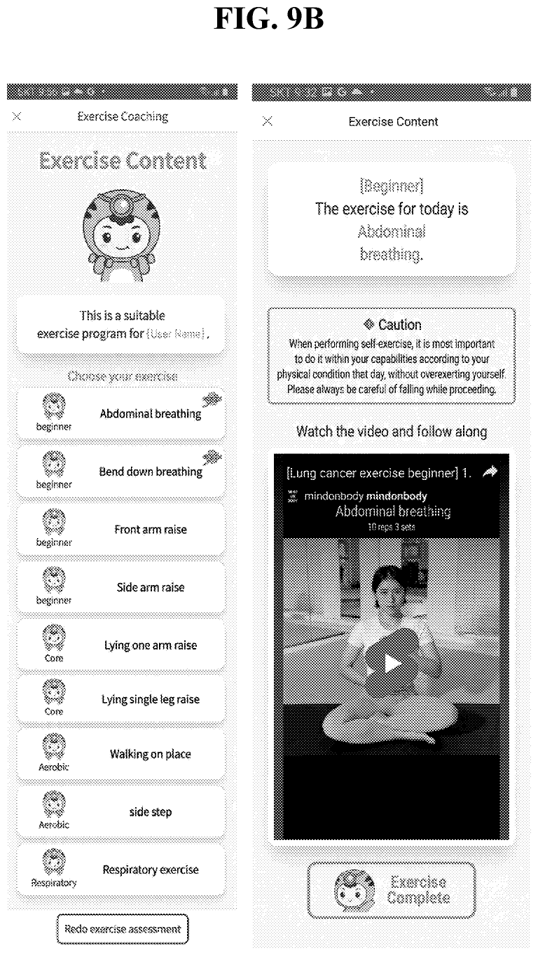

FIGS. 9a and b are diagrams illustrating an example of providing exercise and rehabilitation training according to an embodiment of the present invention.

As shown in FIGS. 9a and 9b, the coaching module (130) can provide appropriate exercise and rehabilitation training based on the user's health status and common cancer type, and provide aerobic exercise and specialized treatments for common cancer types only when necessary.

Here, the coaching module (130) provides exercise and rehabilitation training focused on pelvic floor muscle exercises for colorectal cancer, provides exercise and rehabilitation training focused on breathing exercises and chest exercises for lung cancer, provides exercise and rehabilitation training focused on shoulder and neck exercises for thyroid cancer, and provides exercise and rehabilitation training focused on upper limb exercises for breast cancer.

Additionally, the coaching module (130) can provide Range of Motion (ROM) exercises or Active Assistive Range of Motion (AAROM) exercises when the user's health status is beginner, provide Isometric exercises when the user's health status is intermediate, and provide Strengthening exercises and Stretching exercises when the user's health status is advanced.

The Wellness life design module (140) can provide information on linked institutions (e.g., fitness centers, personal training centers, etc.) within a pre-set radius (e.g., 500 m) based on location information acquired through the user device (100), and can provide dietary information based on user information.

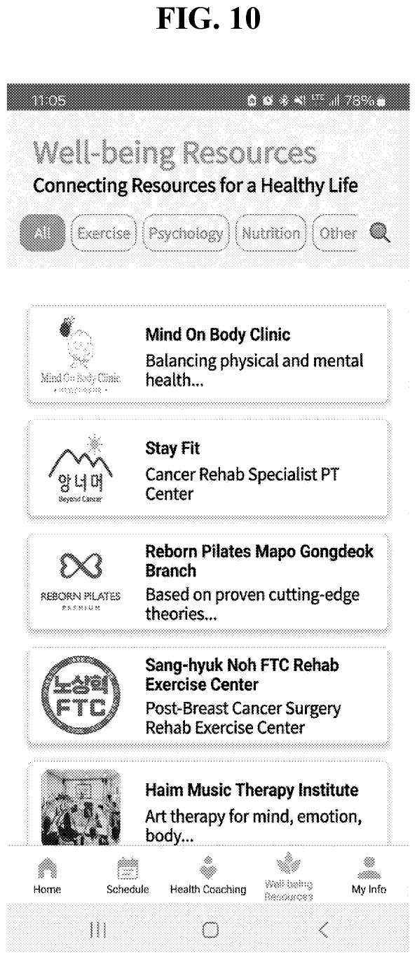

FIG. 10 is a diagram illustrating an example of providing information on linked institutions according to an embodiment of the present invention.

As shown in FIG. 10, the Wellness life design module (140) can provide information on linked institutions (e.g., fitness centers, personal training centers, etc.) within a pre-set radius (e.g., 500 m) based on location information acquired through the user device (100). This linked institution information may include the institution's name, address, contact details, and other relevant data.

Additionally, the user device (100) can transmit at least one of the following to the management server (200): user information, the user's psychological state, the user's health status, the user's sleep status, the user's activity data, the user's dietary habits, and the user's exercise and rehabilitation training history.

Next, the management server (200) can manage multiple user information, monitor each user's psychological state, health status, exercise, and rehabilitation training, and manage the content provided to users.

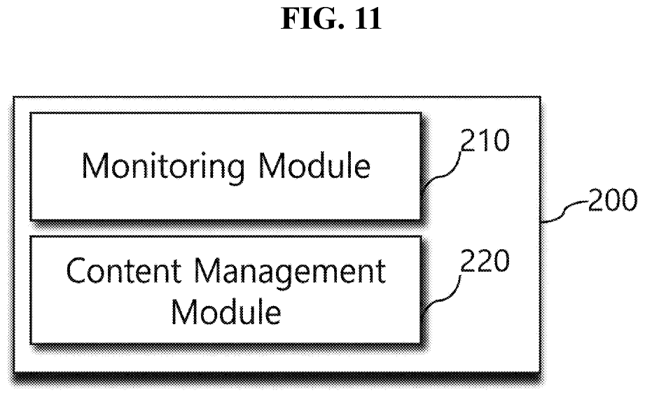

FIG. 11 is a schematic diagram of a management server according to an embodiment of the present invention.

As shown in FIG. 11, the management server (200) can comprise a monitoring module (210) that monitors user information and each user's psychological state, health status, exercise, and rehabilitation training history, and a content management module (220) that manages content provided to users and information from linked institutions.

The monitoring module (210) can input from the user device (100) regarding at least one of the following: user information, the user's psychological state, the user's health status, the user's sleep status, the user's activity data, the user's dietary habits, and the user's exercise and rehabilitation training history, thereby managing comprehensive information for each user.

At this time, the comprehensive information per user can comprise the user's name, date of birth, gender, predominant cancer type, treatment period, exercise and rehabilitation training history, and detailed information. The detailed information can comprise the user's sleep status, dietary habits, activity data, psychological state, health status, and response history regarding their health status.

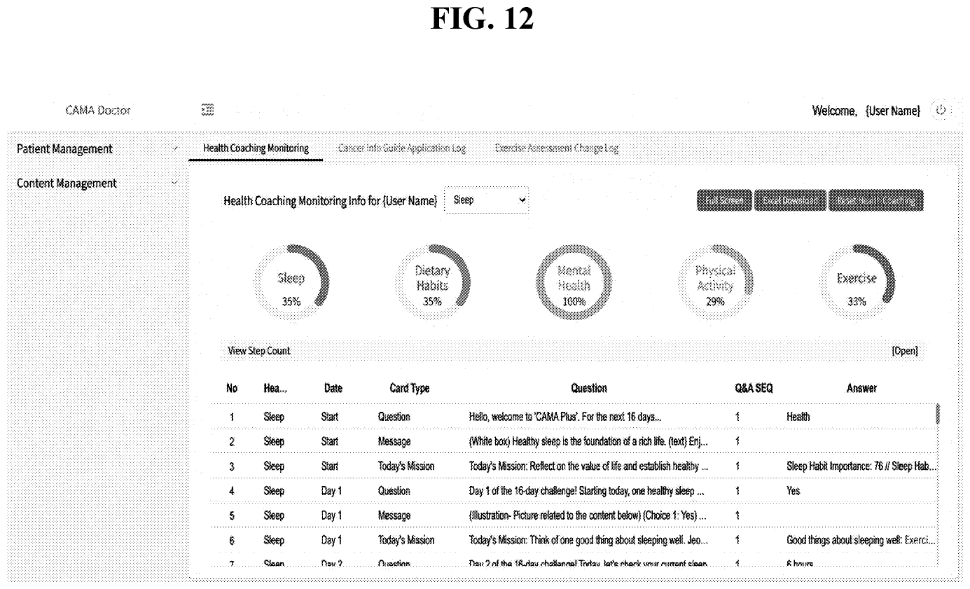

FIG. 12 is a diagram illustrating an example of monitoring a user's health coaching history according to an embodiment of the present invention.

As shown in FIG. 12, the monitoring module (210) can manage the user's responses to health coaching across multiple categories (sleep, dietary habits, mental health, physical activity, exercise) and manage processed questions by matching them.

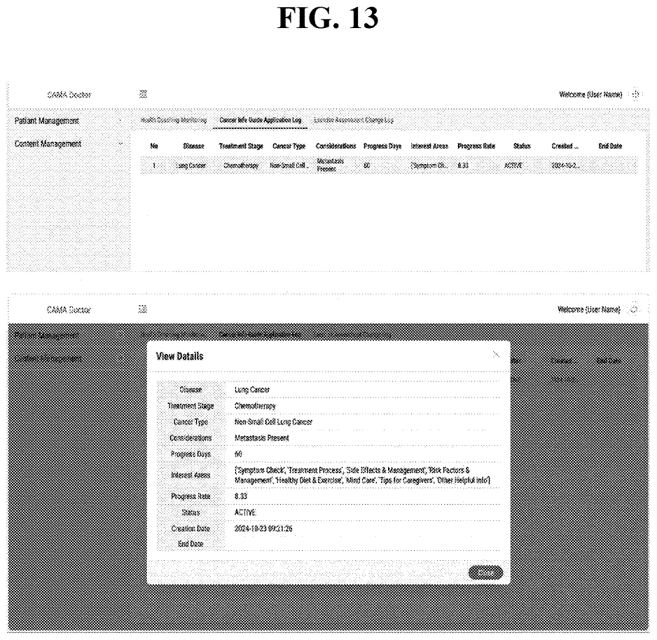

FIG. 13 is a diagram illustrating an example of managing cancer information guide history according to an embodiment of the present invention.

As shown in FIG. 13, the monitoring module (210) enables users to manage their history of applying for cancer information guides.

Additionally, the monitoring module (210) can manage progress rates based on the patient's history of receiving health status evaluations and performing prescribed exercise and rehabilitation training. It provides users with responses regarding exercise and rehabilitation training, and matches these responses with questions to manage them as a history.

The content management module (220) can upload content to be provided to users and modify or delete uploaded content.

Additionally, the content management module (220) can upload information about linked institutions (e.g., name, introduction, address, phone number, website, SNS address, etc.) to be provided based on the user's common cancer type.

According to an embodiment of the present invention, treatment outcomes for cancer patients can be improved by providing alerts for hospital visits, medication intake, and other activities.

Furthermore, adaptive coping with cancer can be fostered by periodically assessing the psychological state of cancer patients at predetermined intervals and providing corresponding coping strategies.

Additionally, personalized treatment guidance for cancer patients can be provided by applying the patient's status responses regarding sleep, dietary habits, physical activity, psychological state, and exercise of cancer patients to a pre-trained algorithm.

The present invention has been described with reference to the embodiments illustrated in the figures, but these are merely illustrative. Those skilled in the art will understand that various modifications and equivalent other embodiments are possible based on the disclosure herein. Therefore, the true scope of technical protection of the present invention should be determined by the technical concepts set forth in the appended claims.

### EXPLANATION OF SYMBOLS

| | |
|---|---|
| 100: User Device | |
| 110: Customer Management Module | |
| 111: Customer Information Storage Unit | 112: Schedule Management Unit |
| 120: Content Provision Module | |
| 130: Coaching Module | |
| 140: Wellness Life Design Module | |
| 200: Management Server | |
| 210: Monitoring Module | |
| 220: Content Management Module | |

What is claimed is:

1. A system for providing customized information and coaching for cancer patients comprises:
a user device that inputs and manages user information from users, provides content and self-management methods to users based on the user information, and designs users' wellness life; and
a management server that manages multiple users' information, monitors each user's psychological state, health status, exercise, and rehabilitation training, and manages the content provided to users,
wherein the user device and management server are a wireless-connected.

2. The system for providing customized information and coaching for cancer patients according to claim 1,
wherein the user device comprises:
a customer management module that inputs and stores user information and the user's cancer treatment schedule from the user;
a content provision module that applies the user information and the user's cancer treatment schedule to a pre-established guide algorithm, considers the common cancer types and treatment timing, and provides content related to at least one of disease, treatment, or side effects;
a coaching module that provides pre-set types of health information based on the user information, assesses the user's health status through pre-set multiple performance evaluation metrics, and provides self-management methods; and
a wellness life design module that provides information on affiliated institutions within a pre-set radius based on the user's location information and provides dietary information based on the user information.

3. The system for providing customized information and coaching for cancer patients according to claim 2,
wherein the user information comprises the user's personal information, the user's common cancer information, and the user's medical information,
wherein the cancer treatment schedule comprises appointment schedules, test schedules, and medication routines,
wherein the user's personal information comprises the user's name, age, gender, and mobile phone number,
wherein the user's common cancer information comprises the type of common cancer type, stage of progression, the user's considerations, content viewing period, treatment timing, and the user's areas of interest,
wherein the user's medical information comprises the user's hospital visit history, prescription history, and surgical history.

4. The system for providing customized information and coaching for cancer patients according to claim 2,
wherein the customer management module comprises:
a customer information storage unit that inputs and stores the user information and the user's cancer treatment schedule from the user; and
a schedule management unit that provides alerts to the user based on the user information and the user's cancer treatment schedule.

5. The system for providing customized information and coaching for cancer patients according to claim 2,
wherein the content provision module provides users with the common cancer guidelines initiated on the server and
content composed of data directly created by evidence-based practices from psychological experts and dietary specialists in the fields of internal medicine, surgery, psychiatry, and psycho-oncology.

6. The system for providing customized information and coaching for cancer patients according to claim 2,
wherein the coaching module evaluates the user's psychological state as one of combat-type, anxious-type, compliance-type, suppression-type or self-abandonment-type using a psychological state assessment scale based on the Mental Adjustment to Cancer Scale (Mini-MAC), and provides pre-set psychological coaching according to the assessed psychological state.

7. The system for providing customized information and coaching for cancer patients according to claim 2,
wherein the coaching module provides appropriate exercise and rehabilitation training based on the user's health status and common cancer types.

8. The system for providing customized information and coaching for cancer patients according to claim 7,
wherein the coaching module provides exercise and rehabilitation training focused on:
pelvic floor muscle exercises for colorectal cancer,
breathing exercises and chest exercises for lung cancer,
shoulder and neck exercises for thyroid cancer,
and upper limb exercises for breast cancer.

9. The system for providing customized information and coaching for cancer patients according to claim 7,
wherein the coaching module:
assesses the user's health status as one of the beginner, intermediate, or advanced using pre-set multiple performance evaluation metric,
provides Range of Motion (ROM) exercises or Active Assistive Range of Motion (AAROM) exercises for users with beginner health status,
provides Isometric exercises for users with intermediate health status,
provides Strengthening exercises and Stretching exercises for users with advanced health status.

10. The system for providing customized information and coaching for cancer patients according to claim 1,
wherein the management server comprises:
a monitoring module that manages comprehensive user information by receiving at least one of the following inputs: user information, user's psychological state, user's health status, user's sleep status, user's activity data, user's dietary habits, and user's exercise and rehabilitation training history; and
a content management module that uploads content to be provided to users and modifies or deletes uploaded content.
